(19) United States
(12) Patent Application Publication
(10) Pub. No.: US 2022/0289534 A1
(43) Pub. Date: Sep. 15, 2022

(54) METHOD FOR ASSISTING IN MAINTAINING A METAL CABLE OF A LIFTING OR TRANSPORT APPARATUS

(71) Applicant: MANITOWOC CRANE GROUP FRANCE, DARDILLY (FR)

(72) Inventors: Bruno RONI-DAMOND, LYON (FR); Jonathan CHALAVON, MIONS (FR); Jérémy BLASIAK, FLEURIEUX SUR L'ARBRESLE (FR); Simon BUCAS, LYON (FR); Sébastien BEYLIER, LIMONEST (FR)

(21) Appl. No.: 17/691,060

(22) Filed: Mar. 9, 2022

(30) Foreign Application Priority Data
Mar. 9, 2021 (FR) 21/02286

(51) Int. Cl. B66D 1/50; B66C 15/00

(52) U.S. Cl. CPC B66D 1/50; B66C 15/00

(57) ABSTRACT

A maintenance assistance method for assisting in maintaining a metal cable of a lifting or transport apparatus, the metal cable forming a closed loop and cooperating with a re-tensioning system. The maintenance assistance method implements the following steps: supervising the re-tensioning operations of the metal cable by the re-tensioning system, for each re-tensioning operation of the metal cable, determining of an elongation value of the metal cable, estimating an overall elongation value of the metal cable, which corresponds to the sum of the elongation values of the metal cable determined after each re-tensioning operation, and determining a damage index of the metal cable as a function of the overall elongation value.

### FIELD

The invention relates to a maintenance assistance method for assisting in a maintenance of a metal cable of a lifting or transport apparatus, and in particular a metal cable which forms a closed loop and which cooperates with a re-tensioning system.

A closed-loop metal cable is a cable which has a closed-loop path and which has a constant length in use, except for the elongations which occur according to the stresses applied on the metal cable. Such a closed-loop metal cable generally has two ends fastened to the same winding drum of a winch, to ensure a displacement of a functional element of the lifting or transport apparatus.

The invention finds a non-limiting application for a crane-type lifting apparatus, and in particular a tower crane, a crane mounted in elements, a self-erecting crane, a port crane and a mobile crane. The invention may also be applied to a cable transport apparatus, operating with a closed-loop metal cable, such as for example a cable car or a chairlift.

### BACKGROUND

In a lifting apparatus application, such as a crane, it is known to use a closed-loop metal cable to displace a distribution trolley in translation along a jib, such a cable being called a distribution cable, and where a load to be lifted is suspended on such a distribution trolley by a lifting cable to make it rise and descent relative to the distribution trolley, thereby allowing a distribution of the load (displacement along the jib) and a lifting of the load (rising and descending of the jib). It should be noted that such a lifting cable does not form a closed loop, but forms an open loop by having only one end fastened to the winding drum of a lifting winch.

In general, metal cables are subject to fatigue damage. The applicant has observed that this fatigue phenomenon is influenced by the level of tension in the metal cable, generally induced by the load, and also by the re-tensioning operations of the metal cable which occur in the lifetime of the metal cable.

Indeed, a closed-loop metal cable generally has a large length, which may exceed one hundred meters depending for example on the lengths of the jib, which helps ensuring that the metal cable extends during the use thereof, so that it is necessary to re-tension this metal cable on a regular basis. In general, it is necessary to perform this re-tensioning of the metal cable about two to three times during the first month of use of the crane, and afterwards about once per quarter. Conventionally, a re-tensioning system, also called tensioning mechanism, is coupled to such a metal cable in a closed loop to ensure this setting of the tension of the metal cable, where this re-tensioning system may for example and without limitation comprise a differential drum device.

Indeed, this re-tensioning of the metal cable in a closed loop is necessary to ensure a proper operation of the displacement of the associated functional element, such as for example the translation of the distribution trolley or the inclination of the lifting jib, which does not permit having a loose metal cable. Also, a cable looseness safety system is sometimes provided, in particular for distribution cables, which comes into action and blocks the distribution trolley in the safety position in accordance with conventional safety requirements, and in particular the indications of the European standard EN 2 14439 entitled “Cranes-Safety-Tower cranes”.

These re-tensioning operations of the metal cable are also necessary to ensure reactive operation of the displacement of the associated functional element and accurate positioning of this functional element. Indeed, at each displacement impulse, the functional element reacts more rapidly when the metal cable is tensioned enough because there would be no or almost no cable looseness to compensate for.

In the case of metal cables, the standards define the limits of damage or wear acceptable for each cable, these limits often being given in number of cut wires observed over a predefined length of the cable. Also, for obvious safety reasons, maintenance operations are regularly carried out to visually inspect the cables in a lifting or transport apparatus, and in particular the metal cables, with however many difficulties: the metal cable may be particularly long and may present sections that are sometimes difficult to access, some types of cables including anti-rotation cables may be damaged with cuts in the interior wires and therefore not visible during an inspection.

### SUMMARY

Thus, although most of the time this type of visual inspection is effective, there is a real need to make reliable and to automate the preventive maintenance of closed loop metal cables to estimate a damage index and thus proceed to the replacement of the weakened cables when necessary.

To this end, the invention suggests a maintenance assistance method for assisting in maintaining a metal cable of a lifting or transport apparatus, said metal cable being a metal cable forming a closed loop and cooperating with a re-tensioning system, said maintenance assistance method implementing the following steps:
supervision of the re-tensioning operations of the metal cable by the re-tensioning system, which occur in the lifetime of the metal cable;
for each re-tensioning operation of the metal cable, determination of an elongation value of the metal cable;
estimation of an overall elongation value of the metal cable, which corresponds to the sum of the elongation values of the metal cable determined after each re-tensioning operation;
determination of a damage index of the metal cable as a function of the overall elongation value.

Thus, the invention is based on an estimation of an overall elongation value of the metal cable to determine the damage index, and if necessary alert an operator; bearing in mind that the invention is based on elongation values after each re-tensioning operation, which is particularly advantageous because these re-tensioning operations are particularly suitable for being able to assess these elongation values, to the extent that re-tensioning aims precisely to take up a cable looseness which results precisely from an elongation of the cable.

It should be noted that, in the context of the invention, the metal cable forming a closed loop may be composed of a single metal cable in a closed loop or of two metal cables which together form a closed loop.

According to one feature, the determination of the damage index of the metal cable is also a function of the number of re-tensioning operations of the metal cable.

Indeed, the number of re-tensioning affects on the damage of the metal cable, and taking it into consideration is particularly advantageous in the context of this preventive supervision.

According to another feature, the determination of the damage index of the metal cable is also a function of the time intervals between the successive re-tensioning operations of the metal cable.

Indeed, several re-tensioning operations in a reduced time interval, for example within few days, would show an anomaly, and therefore a potential damage which will be taken into consideration in establishing the damage index.

According to one possibility, the method comprises, after each re-tensioning operation of the metal cable, a comparison of the damage index of the metal cable with a predefined damage threshold, and an issuing of an alert if the damage index of the metal cable exceeds said damage threshold.

According to one possibility, the damage index corresponds to the overall elongation value, and the damage threshold corresponds to an elongation maximum threshold.

In other words, it is the overall elongation value which will be used for the damage index.

According to one feature, the metal cable has two ends fastened to the same winding drum of a winch, to ensure a displacement of a functional element of the lifting or transport apparatus.

Such a metal cable may for example be composed of two strands which are wound on the same winding drum and which are fastened on the same functional element. Thus, each strand has a first end fastened to the winding drum, and a second end fastened to the functional element. The first ends of the two strands thus form the ends of the metal cable, and these two strands form properly a closed loop.

In a particular embodiment, the metal cable is a distribution metal cable ensuring a displacement of a distribution trolley displaceable along a jib of a lifting or transport apparatus.

Such a distribution metal cable is generally composed of two strands which are wound on the same winding drum and which are fastened on the same distribution trolley to form a closed loop.

Advantageously, the supervision of the re-tensioning operations of the distribution metal cable implements:
a supervision of a distribution winch comprising a winding drum on which a rear strand and a front strand of the distribution metal cable are wound to displace the distribution trolley, said supervision of the distribution winch comprising a determination of a theoretical position of the distribution trolley as a function of a measure of winding of the rear strand or of the front strand on the winding drum; and
a supervision of an actual position of the distribution trolley on the jib;

and wherein, for each re-tensioning operation of the distribution metal cable, the determination of an elongation value of the distribution metal cable consists in establishing the elongation value of the distribution metal cable as corresponding to the difference between the theoretical position of the distribution trolley and the actual position of the distribution trolley.

Thus, the elongation values will be determined by comparing, during each re-tensioning operation, the theoretical position of the distribution trolley and the actual position of the distribution trolley. The theoretical position of the distribution trolley corresponds to the position that the distribution trolley should have if the distribution metal cable had not lengthened since the previous re-tensioning, and this theoretical position is established on the basis of a measure of winding of the rear strand and/or the front strand on the drum. The actual position of the distribution trolley corresponds to the real position of the distribution trolley, which may be measured by means of a sensor placed on the jib and/or on the distribution trolley.

Advantageously, after each re-tensioning operation of the distribution metal cable, a resetting of the theoretical position of the distribution trolley is implemented in order to calibrate this theoretical position of the distribution trolley to the actual position of the distribution trolley.

Thus, this resetting allows automatically re-tracking the “zero range” of the distribution trolley which corresponds to the reference position of the distribution trolley to determine its position along the jib. After the automation of this resetting, the risk of error on this reference position is eliminated and the range indication will always remain correct and reliable.

According to one possibility, the alert is in the form of a visual or audible alert signal on a control interface, such as for example a control interface for the crane operator or for a remote supervision service.

The invention also relates to a lifting or transport apparatus comprising a metal cable which forms a closed loop and which cooperates with a re-tensioning system, said crane comprising:
a supervision system for supervising re-tensioning operations of the metal cable by the re-tensioning system, which occur in the lifetime of the metal cable;
a monitoring/control system configured to:
determine an elongation value of the metal cable after each re-tensioning operation of the metal cable;
estimate an overall elongation value of the metal cable, which corresponds to the sum of the elongation values of the metal cable determined after each re-tensioning operation;
determine a damage index of the metal cable as a function of the overall elongation value.

In a particular embodiment, the lifting or transport apparatus comprises a winch provided with a winding drum and the metal cable has two ends fastened to this winding drum, to ensure a displacement of a functional element of the lifting or transport apparatus.

In a particular embodiment, the metal cable is a distribution metal cable ensuring a displacement of a distribution trolley displaceable along a jib of the lifting or transport apparatus.

According to one possibility, the monitoring/control system is configured to determine the damage index of the metal cable also as a function of the number of re-tensioning operations of the metal cable.

According to another possibility, the monitoring/control system is configured to determine the damage index of the metal cable also as a function of the time intervals between the successive re-tensioning operations of the metal cable.

In an advantageous embodiment, the lifting or transport apparatus comprises an alert system connected to the monitoring/control system, said monitoring/control system being configured to, after each re-tensioning operation of the metal cable, compare the damage index of the metal cable with a predefined damage threshold, and to activate the alert system to issue an alert if the damage index of the metal cable exceeds said damage threshold.

### BRIEF DESCRIPTION OF THE DRAWINGS

Other features and advantages of the present invention will appear upon reading the detailed description hereinafter, of a non-limiting example of implementation, made with reference to the appended figures in which:

FIG. 1 is a schematic view of a crane according to the invention;

FIG. 2 is a schematic view of a sequence of steps implemented in the maintenance assistance method according to the invention.

### DESCRIPTION

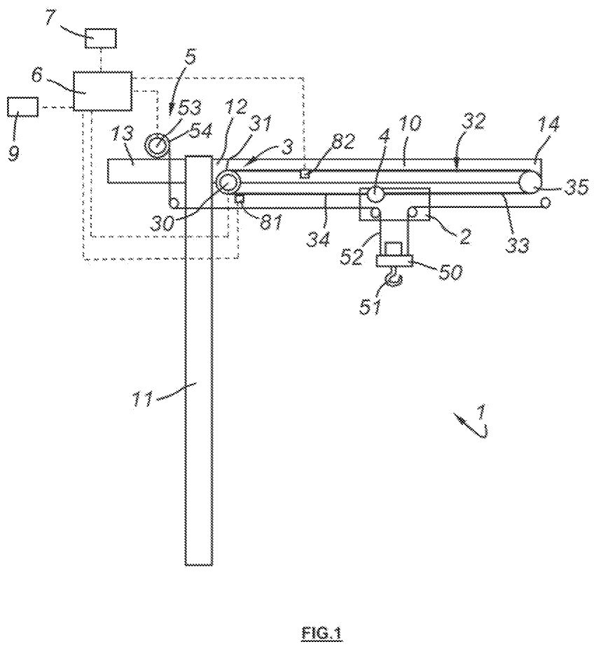

Referring to FIG. 1, a crane 1 according to the invention, for example of the tower crane type, comprises a distribution jib 10 mounted on a tower 11 (also called a mast) at the root 12 of the jib 10. Conventionally, the root 12 of the jib 10 is rotatably mounted on the tower 11 according to a vertical axis. The jib 10 may be extended to the other side of the tower 11 by a counter-jib 13, generally provided with ballast. Thus, the jib 10 and the counter-jib 13 form a rotating portion fastened to a pivot, also called the orientation member, making the connection between this rotating portion and the tower 11; such a pivot incorporating a motor-driven orientation system to actuate the rotation of the rotating portion along the vertical axis.

The crane 1 further includes a distribution trolley 2 designed so as to distribute a load (not illustrated) along the jib 10, this distribution trolley 2 circulating on a rolling path formed on the jib 10, between the root 12 and the tip 14 of the jib 10, also called free end of the jib 10.

This distribution trolley 2 is connected to a distribution winch 3 suitable for displacing the distribution trolley 2 along the roller track in a forward direction (in other words in the direction of the tip 14 of the jib 10, on the right of the figure) and a backward direction (in other words in the direction of the root 12 of the jib 10, on the left of the figure) that are opposite to each other. This distribution winch 3 is preferably disposed on the jib 10.

As schematized, this distribution winch 3 comprises a distribution motor 30 driving a winding drum 31 coupled to a distribution metal cable 32 having these two ends fastened on this winding drum 31. This distribution metal cable 32 thus has a front strand 33 (also called front cable) and a rear strand 34 (also called rear cable) fastened on either side of the distribution trolley 2. The front strand 33 and the rear strand 34 are both wound on the winding drum 31, and the front strand 33 circulates from the winding drum 31 to a front pulley 35 disposed on the tip 14 of the jib 10 before returning to the front of the distribution trolley 2, whereas the rear strand 34 circulates from the winding drum 31 to the rear of the distribution trolley 2. Thus, the distribution metal cable 32 is formed of the front strand 33 and the rear strand 34 which are two metal cables wound on the same winding drum 31 and fastened on the same distribution trolley 2 to form together a closed loop.

The front strand 33 and/or the rear strand 34 of the distribution metal cable 32 is or are coupled to a re-tensioning system 4 carried by the distribution trolley 2. This re-tensioning system 4 forms a re-tensioning mechanism with manual or automated actuation, which is coupled to the distribution metal cable 32 to ensure a setting of the tension of the distribution metal cable 32. As a non-limiting example, this re-tensioning system 4 is a differential drum device. This differential drum device may for example be used to “shorten” the front strand 33 only (or the rear strand 34 only) while contributing to the fact that the two strands, front 33 and rear 34, are re-tensioned at the same time because they form together a closed loop. This distribution metal cable 32 thus forms a closed loop, its length remaining constant during the displacements of the distribution trolley 2, except for the elongations which occur as and when the stresses on the distribution metal cable 32 are applied.

This distribution trolley 2 also supports pulleys which guide a lifting cable 52 which supports a reeve-block device 50, which is thus suspended from the distribution trolley 2 by the lifting cable 52. This reeve-block device 50 supports a lifting member 51 provided for hooking of the load and which may be in the form of a hook articulated on the reeve-block device 50. The crane 1 comprises a lifting winch 5 provided with a lifting motor 53 driving a lifting drum 54 on which is fastened only one end of the lifting cable 52. This lifting cable 52 therefore passes over the pulleys of the distribution trolley 2 and circulates to the tip 14 of the jib 10. This lifting cable 52 thus forms an open loop, its length being variable by winding/unwinding around the lifting drum 54 to enable the load to be lifted/lowered.

The crane 1 comprises a monitoring/control system 6 which is connected to the distribution winch 3 and to the lifting winch 5 for a control of the motor speed and the motor direction of the distribution motor 30, in front and rear distribution, and also of the lifting motor 53, in elevation and in lowering, and thus for a control of the displacement of the load. This monitoring/control system 6 is also connected to a pilot device 7, used to pilot the functions of the crane 1, by a pilot or crane operator, so that the monitoring/control system 6 receives instructions from the pilot device 7 to control the motors 30, 53.

This monitoring/control system 6 is connected to a supervision system for supervising re-tensioning operations of the distribution metal cable 32 by the re-tensioning system 4, which intervene in the lifetime of the distribution metal cable 32.

This supervision system comprises a first sensor 81 positioned at the level of the winding drum 31 of the distribution winch 3 to detect a measure of winding of the rear strand 34 or of the front strand 33 on the winding drum 31. This first sensor 81 thus makes it possible to carry out a supervision of the distribution winch 3 by the implementation of a determination of a theoretical position XTH of the distribution trolley 2 as a function of such a measure of winding of the rear strand 34 or of the front strand 33 on the winding drum 31.

This supervision system comprises a second sensor 82 positioned on the jib 10 along the distribution metal cable 32, for example at the level of the root 12 of the jib 10, to detect an actual position XRL of the distribution trolley 2.

After re-tensioning the distribution metal cable 32, the monitoring/control system 6 performs a calibration or a resetting of the theoretical position XTH of the distribution trolley 2 in order to calibrate this theoretical position XTH of the distribution trolley 2 on the actual position XRL of the distribution trolley, in other words upon completion of this resetting XTH=XRL. The monitoring/control system 6 thus follows each re-tensioning operation of the distribution metal cable 32, by the occurrence of a readjustment of the theoretical position XTH.

This monitoring/control system 6 is configured to:
after each re-tensioning operation of the distribution metal cable 32, determine an elongation value ΔL of distribution metal cable 32 corresponding to the difference between the theoretical position XTH of the distribution trolley and the actual position XRL of the distribution trolley 2 before the re-tensioning operation, in other words ΔL=XRL−XTH;
estimate an overall elongation value ΔLG of the distribution metal cable 32, which corresponds to the sum of the elongation values ΔL of the distribution metal cable 32 determined after each re-tensioning operation;
determine a damage index IND of the distribution metal cable 32 according to the overall elongation value ΔLG.

It should be noted that the damage index IND may also be calculated as a function of the number of re-tensioning operations of the distribution metal cable 32 and/or the time intervals between the successive re-tensioning operations of the distribution metal cable 32 (in other words the frequency of re-tensioning operations). Furthermore, this damage index IND may correspond directly to the overall elongation value ΔLG, i.e. IND=ΔLG, with optionally weightings associated with the number of re-tensioning operations and/or the time intervals between successive re-tensioning operations.

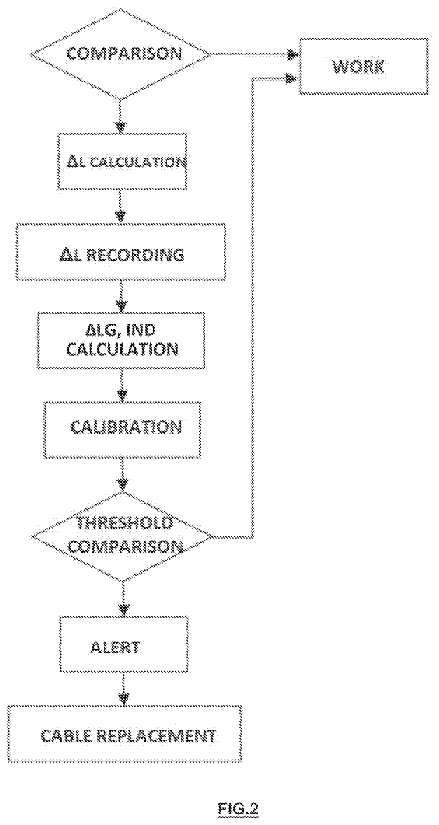

FIG. 2 illustrates the steps implemented for this crane 1 for a method of assisting in maintaining the distribution metal cable 32, that begins with a “COMPARISON” step which consists in comparing the theoretical position XTH and the actual position XRL, if the theoretical position XTH corresponds to the actual position XRL (i.e. XTH=XRL) then charge lifting/distribution work operations are possible in a “WORK” phase, on the other hand if the theoretical position XTH does not correspond to the actual position XRL (i.e. XTH≠XRL) then a phase of re-tensioning and of determination of the damage index IND is initiated.

The phase of re-tensioning and of determination of the damage index IND begins with a “ΔL CALCULATION” step which consists in calculating the elongation value ΔL which is equal to the difference between the theoretical position XTH and the actual position XRL, followed by a “ΔL RECORDING” step which consists in recording the elongation value ΔL in a memory of the monitoring/control system 6. It should be noted that these elongation values ΔL are deleted or reset during a replacement of the distribution metal cable 32.

Afterwards, a “ΔLG, IND CALCULATION” step consists in calculating the overall elongation value ΔLG which corresponds to the sum of the elongation values ΔL recorded in the memory, and then in calculating the damage index IND as a function of this overall elongation value ΔLG. Then, a “CALIBRATION” step consists in calibrating the theoretical position XTH to correspond to the actual position XRL (i.e. XTH=XRL). Then a “THRESHOLD COMPARISON” step which consists in comparing the damage index IND with a predefined damage threshold LIM. If the damage index IND is smaller than the damage threshold LIM (which means that the damage index IND is low and the cable is still usable) then the load lifting/distribution work operations are possible in the “WORK” phase, on the other hand if the damage index IND is greater than the damage threshold LIM (which means that the damage index IND is high and the cable is too damaged to be usable) then an “ALERT” step is implemented, which consists in issuing an alert to warn an operator, such as the crane operator, of the situation and triggering afterwards a “CABLE REPLACEMENT” phase consisting in removing the distribution metal cable 32 to replace it with a new.

Thus, the crane 1 comprises an alert system 9 connected to the monitoring/control system 6 and configured to issue an alert when activated. This alert system 9 may be in the form of a visual display, for example at the level of a control interface placed in a control cabin or of a remote interface, so that the alert signal is a visual signal on this control interface. Alternatively or complementarily, this alert system 9 may comprise a sound transmitter, for example in the control cabin, so that the alert signal is a sound signal.

The invention described hereinabove is therefore applied to a distribution metal cable 32 of a crane, but it may also be applied to other types of closed-loop metal cable and/or to other types of cable-operated lifting or transport apparatus.

1-17. (canceled)

18. A maintenance assistance method for assisting in maintaining a metal cable of a lifting or transport apparatus, the metal cable being a metal cable forming a closed loop and cooperating with a re-tensioning system, the maintenance assistance method implementing the following steps:
supervision of re-tensioning operations of the metal cable by the re-tensioning system, which occur in the lifetime of the metal cable;
for each operation of re-tensioning the metal cable, determination of an elongation value of the metal cable;
estimation of an overall elongation value of the metal cable, which corresponds to the sum of the elongation values of the metal cable determined after each re-tensioning operation; and
determination of a damage index of the metal cable as a function of the overall elongation value.

19. The maintenance assistance method according to claim 18, wherein the determination of the damage index of the metal cable depends also on the number of re-tensioning operations of the metal cable.

20. The maintenance assistance method according to claim 18, wherein the determination of the damage index of the metal cable is also a function of the time intervals between the successive re-tensioning operations of the metal cable.

21. The maintenance assistance method according to claim 18, comprising, after each re-tensioning operation of the metal cable, a comparison of the damage index of the metal cable with a predefined damage threshold, and an issuing of an alert if the damage index of the metal cable exceeds the damage threshold.

22. The maintenance assistance method according to claim 21, wherein the damage index corresponds to the overall elongation value, and the damage threshold corresponds to an elongation maximum threshold.

23. The maintenance assistance method according to claim 21, wherein the alert is in the form of a visual or audible alert signal on a control interface.

24. The maintenance assistance method according to claim 18, wherein the metal cable has two ends fastened to a same winding drum of a winch, for ensuring a displacement of a functional element of the lifting or transport apparatus.

25. The maintenance assistance method according to claim 18, wherein the metal cable is a distribution metal cable ensuring a displacement of a distribution trolley displaceable along a jib of the lifting or transport apparatus.

26. The maintenance assistance method according to claim 25, wherein the supervision of the re-tensioning operations of the distribution metal cable implements:
a supervision of a distribution winch comprising a winding drum on which are wound a rear strand and a front strand of the distribution metal cable to displace the distribution trolley, the supervision of the distribution winch comprising a determination of a theoretical position of the distribution trolley as a function of a measure of winding of the rear strand or of the front strand on the winding drum; and
a supervision of an actual position of the distribution trolley on the jib,
wherein, for each re-tensioning operation of the distribution metal cable, the determination of an elongation value of the distribution metal cable consists in establishing the elongation value of the distribution metal cable as corresponding to the difference between the theoretical position of the distribution trolley and the actual position of the distribution trolley.

27. The maintenance assistance method according to claim 26, wherein, after each re-tensioning operation of the distribution metal cable, a resetting of the theoretical position of the distribution trolley is implemented in order to calibrate this theoretical position of the distribution trolley on the actual position of the distribution trolley.

28. A lifting or transport apparatus comprising a metal cable which forms a closed loop and which cooperates with a re-tensioning system, the lifting or transport apparatus comprising:
a supervision system for supervising re-tensioning operations of the metal cable by the re-tensioning system, which occur in the lifetime of the metal cable;
a monitoring/control system configured to:
determine an elongation value of the metal cable after each re-tensioning operation of the metal cable;
estimate an overall elongation value of the metal cable, which corresponds to the sum of the elongation values of the metal cable determined after each re-tensioning operation; and
determine a damage index of the metal cable as a function of the overall elongation value.

29. The lifting or transport apparatus according to claim 28, comprising a winch provided with a winding drum and the metal cable has two ends fastened to this winding drum, to ensure a displacement of a functional element of the lifting or transport apparatus.

30. The lifting or transport apparatus according to claim 29, wherein the metal cable is composed of two strands which are wound on the winding drum and which are fastened on the same functional element.

31. The lifting or transport apparatus according to claim 29, wherein the metal cable is a distribution metal cable ensuring a displacement of a distribution trolley displaceable along a jib of the lifting or transport apparatus.

32. The lifting or transport apparatus according to claim 28, wherein the monitoring/control system is configured to determine the damage index of the metal cable as a function of the number of re-tensioning operations of the metal cable.

33. The lifting or transport apparatus according to claim 28, wherein the monitoring/control system is configured to determine the damage index of the metal cable also as a function of the time intervals between the successive re-tensioning operations of the metal cable.

34. The lifting or transport apparatus according to claim 28, comprising an alert system connected to the monitoring/control system, the monitoring/control system being configured to, after each re-tensioning operation of the metal cable, compare the damage index of the metal cable with a predefined damage threshold, and to activate the alert system in order to issue an alert if the damage index of the metal cable exceeds the damage threshold.
